(19) United States
(12) Patent Application Publication
(10) Pub. No.: US 2013/0330283 A1
(43) Pub. Date: Dec. 12, 2013

(54) ORAL CARE COMPOSITIONS

(71) Applicants: Robert Vogt, Princeton, NJ (US); Karsten Kohrs, Berkeley Heights, NJ (US); Steven Wade Fisher, Middlesex, NJ (US); Thomas Campbell, Flemington, NJ (US); Michael Prencipe, Princeton Junction, NJ (US)

(72) Inventors: Robert Vogt, Princeton, NJ (US); Karsten Kohrs, Berkeley Heights, NJ (US); Steven Wade Fisher, Middlesex, NJ (US); Thomas Campbell, Flemington, NJ (US); Michael Prencipe, Princeton Junction, NJ (US)

(73) Assignee: COLGATE-PALMOLIVE COMPANY, New York, NY (US)

(21) Appl. No.: 13/985,536

(22) PCT Filed: Feb. 15, 2011
(86) PCT No.: PCT/US11/24874

§ 371 Date: Aug. 14, 2013

(51) Int. Cl. A61K 8/97; A61Q 11/00; A61K 8/33; A61K 8/49; A61K 8/42; A61K 8/34

(52) U.S. Cl. CPC A61K 8/97; A61K 8/42; A61K 8/34; A61K 8/33; A61K 8/347; A61K 8/498; A61Q 11/00
USPC 424/54; 424/58; 424/49

(57) ABSTRACT

Flavour components for use in oral care compositions containing a metal salt, compositions comprising the flavour components, and methods of making and using the same. The flavour components comprise a taste -masking agent comprising cinnamic aldehyde, eugenol and eucalyptol and one or more flavouring agents selected from L-menthol, N-ethyl-p-menthane-3-carboxamide, anethole, peppermint oil, spearmint oil and corn mint oil. In particular the metal salt is zinc citrate.

### BACKGROUND

There is an ongoing need to improve the taste profile of oral care compositions containing metal salts.

### SUMMARY

Some embodiments of the present invention provide a flavor component for use in an oral care composition containing a metal salt, comprising: a taste-masking agent; and one or more flavoring agents.

Other embodiments provide compositions comprising any one of the flavor components described herein. Further embodiments provide methods for preparing and using the flavor components and compositions described herein.

### DETAILED DESCRIPTION

As used throughout, ranges are used as shorthand for describing each and every value that is within the range. Any value within the range can be selected as the terminus of the range.

All references cited herein are hereby incorporated by reference in their entireties.

In the event of a conflict in a definition in the present disclosure and that of a cited reference, the present disclosure controls.

In some embodiments, the present invention provides a flavor component for use in an oral care composition containing a metal salt, comprising: a taste-masking agent comprising: cinnamic aldehyde; eugenol; and eucalyptol; and one or more flavoring agents selected from: L-menthol; N-ethyl-para-menthan-3-carboxamide; anethole; peppermint oil; spearmint oil; corn mint oil; and a combination of two or more thereof.

In some embodiments, at least one of the one or more flavoring agents is L-menthol. In some embodiments, at least one of the one or more flavoring agents is N-ethyl-para-menthan-3-carboxamide. In further embodiments, at least one of the one or more flavoring agents is anethole. In other embodiments, at least one of the one or more flavoring agents is peppermint oil. Still other embodiments provide a flavor component wherein at least one of the one or more flavoring agents is spearmint oil. While other embodiments provide a flavor component wherein at least one of the one or more flavoring agents is corn mint oil.

Some embodiments comprise greater than 29.5%, by weight, L-menthol. Other embodiments comprise greater than 4.5%, by weight, N-ethyl-para-menthan-3-carboxamide. Further embodiments comprise less than 14.5%, by weight, anethole. In some embodiments, the flavor component comprises less than 40.5%, by weight, peppermint oil. Yet other embodiments comprise less than 5.5%, by weight, spearmint oil. Some embodiments comprise greater than 4.5%, by weight, corn mint oil.

In some embodiments, the present invention provides a flavor component for use in an oral care composition containing a metal salt, comprising: L-menthol; N-ethyl-para-menthan-3-carboxamide; anethole; cinnamic aldehyde; eugenol; eucalyptol; peppermint oil; spearmint oil; and corn mint oil.

Some embodiments provide a flavor component comprising: greater than 29.5%, by weight, L-menthol; greater than 4.5%, by weight, N-ethyl-para-menthan-3-carboxamide; less than 14.5%, by weight, anethole; greater than 0.1%, by weight, cinnamic aldehyde; greater than 0.1%, by weight, eugenol; greater than 0.5%, by weight, eucalyptol; less than 40.5%, by weight, peppermint oil; less than 5.5%, by weight, spearmint oil; and greater than 4.5%, by weight, corn mint oil.

In some embodiments, the flavor component comprises: from about 30 to about 50%, by weight, L-menthol; from about 5 to about 10%, by weight, N-ethyl-para-menthan-3-carboxamide; from about 5 to about 14%, by weight, anethole; from about 0.1 to about 1%, by weight, cinnamic aldehyde; from about 0.1 to about 1%, by weight, eugenol; from about 1 to about 5%, by weight, eucalyptol; from about 20 to about 40%, by weight, peppermint oil; from about 1 to about 5%, by weight, spearmint oil; and from about 5 to about 10%, by weight, corn mint oil.

Some embodiments provide a flavor component comprising: from about 35 to about 45%, by weight, L-menthol; from about 5 to about 8%, by weight, N-ethyl-para-menthan-3-carboxamide; from about 8 to about 12%, by weight, anethole; from about 0.5 to about 0.9%, by weight, cinnamic aldehyde; from about 0.5 to about 0.9%, by weight, eugenol; from about 2 to about 4%, by weight, eucalyptol; and from about 22 to about 27%, by weight, peppermint oil.

Some embodiments provide an oral care composition comprising: any one of the flavor components described herein; one or more metal salts selected from: a zinc salt; a calcium salt; a copper salt; an iron salt; a magnesium salt; a manganese salt; and a combination of two or more thereof; and an orally acceptable carrier.

In some embodiments, at least one of the one or more metal salts is a zinc salt selected from: zinc oxide; zinc sulfate; zinc chloride; zinc citrate; zinc lactate; zinc gluconate; zinc malate; zinc tartrate; zinc carbonate; zinc phosphate; and a combination of two or more thereof. In some embodiments, the zinc salt is selected from: zinc oxide; zinc citrate; zinc gluconate; zinc lactate; and a combination of two or more thereof. In other embodiments, the zinc salt is selected from: zinc oxide; zinc citrate; and a combination of two or more thereof. In some embodiments, the zinc salt is zinc citrate.

In some embodiments, the metal salt is present at a concentration of from about 0.01% to about 5%, by weight of the composition. In other embodiments, the metal salt is present at a concentration of from about 0.1 to about 4%, by weight of the composition. While other embodiments provide compositions wherein the metal salt is present at a concentration of from about 1 to about 3%, by weight. In some embodiments, the metal salt is present at a concentration of about 2%, by weight of the composition.

In some embodiments, the flavor oil component is present at a concentration of from about 0.5 to about 2.5%, by weight. Some embodiments provide a composition wherein the flavor oil component is present at a concentration of from about 0.8 to about 1.5%, by weight. Other embodiments provide a composition wherein the flavor oil component is present at a concentration of about 1%, by weight.

In some embodiments, the compositions further comprise one or more components selected from: a fluoride ion source; a tartar control agent; a buffering agent; an abrasive; and a combination of two or more thereof. In some embodiments, at least one of the one or more components is a fluoride ion source selected from: stannous fluoride, sodium fluoride, potassium fluoride, sodium monofluorophosphate, sodium fluorosilicate, ammonium fluorosilicate, amine fluoride, ammonium fluoride, and a combination of two or more thereof.

The amount of the flavor component included in a metal salt containing composition is generally and functionally described as an amount effective to mask the taste of the metal salt. In some embodiments, the flavor component mitigates the negative attributes of the metal salts without detracting from consumer acceptance of the product.

Other optional additives may be included. Among such optional additives, included are those provided in order to change appearance or aesthetic appeal, and/or to preservative the final product, and/or for taste/cosmetic appeal and/or as therapeutic and prophylactic ingredients for oral health, prevention or treatment of a condition or disorder of hard or soft tissue of the oral cavity, or the prevention or treatment of a physiological disorder or condition.

Colorants such as dyes may be food color additives presently certified under the Food Drug & Cosmetic Act for use in food and ingested drugs, including dyes such as FD&C Red No. 3 (sodium salt of tetraiodofluorescein), Food Red 17, disodium salt of 6-hydroxy-5-{(2-methoxy-5-methyl-4-sulphophenyl)azo}-2-naphthalenesulfonic acid, Food Yellow 13, sodium salt of a mixture of the mono and disulphonic acids of quinophtalone or 2-(2-quinolyl) indanedione, FD&C Yellow No. 5 (sodium salt of 4-p-sulfophenylazo-1-p-sul-fophenyl-5-hydroxypyrazole-3 carboxylic acid), FD&C Yellow No. 6 (sodium salt of p-sulfophenylazo-B-naphtol-6-monosulfonate), FD&C Green No. 3 (disodium salt of 4-{[4-(N-ethyl-p-sulfobenzylamino)-phenyl]-(4-hydroxy-2-sulfoniumphenyl)-methylene}-[1-(N-ethyl-N-p-sulfobenzyl)-.DELTA.-3,5-cycl-ohexadienimine], FD&C Blue No. 1 (disodium salt of dibenzyldiethyl-diamino-triphenylcarbinol trisulfonic acid anhydrite), FD&C Blue No. 2 (sodium salt of disulfonic acid of indigotin) and mixtures thereof in various proportions. Typically, colorants if included are present in very small quantities.

Sweeteners include both natural and artificial sweeteners. Suitable sweetener include water soluble sweetening agents such as monosaccharides, disaccharides and polysaccharides such as xylose, ribose, glucose (dextrose), mannose, galactose, fructose (levulose), sucrose (sugar), maltose, water soluble artificial sweeteners such as the soluble saccharin salts, i.e., sodium or calcium saccharin salts, cyclamate salts dipeptide based sweeteners, such a L-aspartic acid derived sweeteners, such as L-aspartyl-L-phenylalaine methyl ester (aspartame). In general, the effective amount of sweetener is utilized to provide the level of sweetness desired for a particular composition, will vary with the sweetener selected. This amount will normally be about 0.001% to about 5% by weight of the composition. In some embodiments, the sweetener is sodium saccharin and present at about 0.01% by weight of the composition.

Optional breath freshening agents may be provided. Any orally acceptable breath freshening agent can be used, including without limitation zinc salts such as zinc gluconate, zinc citrate and zinc chlorite, alpha-ionone and mixtures thereof. One or more breath freshening agents are optionally present in a breath freshening effective total amount.

Optionally, the composition may include a tartar control (anticalculus) agent. Tartar control agents among those useful herein include phosphates and polyphosphates (for example pyrophosphates), polyaminopropanesulfonic acid (AMPS), polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids and salts of any of these agents, for example their alkali metal and ammonium salts. Useful inorganic phosphate and polyphosphate salts include monobasic, dibasic and tribasic sodium phosphates, sodium tripolyphosphate, tetrapolyphosphate, mono-, di-, tri- and tetrasodium pyrophosphates, sodium trimetaphosphate, sodium hexametaphosphate and mixtures thereof, wherein sodium can optionally be replaced by potassium or ammonium. Other useful anticalculus agents include polycarboxylate polymers and polyvinyl methyl ether/maleic anhydride (PVME/MA) copolymers, such as those available under the Gantrez™ brand from ISP, Wayne, N.J.

In some embodiments, tartar control agent is present at a concentration of from about 0.01 to 10%, by weight. In some embodiments, the tartar control agent is present at a concentration of about 1%, by weight. In some embodiments, sodium phosphate monobasic is present at a concentration of from about 0.01 to about 5%, by weight. In some embodiments, sodium phosphate monobasic is present at a concentration of about 1%, by weight. In some embodiments, disodium phosphate is present at a concentration of from about 0.01 to about 5%, by weight. In some embodiments, disodium phosphate is present at a concentration of about 0.15%, by weight.

Other optional additives include antimicrobial (e.g., antibacterial) agents. Any orally acceptable antimicrobial agent can be used, including triclosan (5-chloro-2-(2,4-dichlorophenoxy)phenol); zinc and stannous ion sources; quaternary ammonium compounds such as cetylpyridinium chloride (CPC); bisguanides such as chlorhexidine; and benzalkonium chloride. A further illustrative list of useful antibacterial agents is provided in U.S. Pat. No. 5,776,435 to Gaffar, et al.

Antioxidants are another class of optional additives. Any orally acceptable antioxidant can be used, including butylated hydroxyanisole (BHA), butylated hydroxytoluene (BHT), vitamin A, carotenoids, vitamin E, flavonoids, polyphenols, ascorbic acid, herbal antioxidants, chlorophyll, melatonin, and mixtures thereof.

Also optional, a saliva stimulating agent, useful for example in amelioration of dry mouth may be included. Any orally acceptable saliva stimulating agent can be used, including without limitation food acids such as citric, lactic, malic, succinic, ascorbic, adipic, fumaric, and tartaric acids, and mixtures thereof. One or more saliva stimulating agents are optionally present in a saliva stimulating effective total amount.

Optionally, an antiplaque (e.g., plaque disrupting) agent may be included. Any orally acceptable antiplaque agent can be used, including without limitation stannous, copper, magnesium and strontium salts, dimethicone copolyols such as cetyl dimethicone copolyol, papain, glucoamylase, glucose oxidase, urea, calcium lactate, calcium glycerophosphate, strontium polyacrylates and mixtures thereof.

Optional desensitizing agents include potassium citrate, potassium chloride, potassium tartrate, potassium bicarbonate, potassium oxalate, potassium nitrate, strontium salts, and mixtures thereof.

Optionally, an inorganic or a natural or synthetic thickener or gelling agent may be present. In some embodiments, the thickener or gelling agent is present in the amount of from about 0.10 to about 5% by weight, or from about 0.2 to about 1% by weight. Suitable thickeners or gelling agents useful in the practice of the present invention include, for example and not limitation, inorganic thickening silicas such as amorphous silicas available from Huber Corporation under the trade designation Zeodent 165, Irish moss, iota-carrageenan, gum tragacanth, and polyvinylpyrrolidone.

In some embodiments, the compositions include a dental abrasive or combination of dental abrasive agents known in the art. Abrasives suitable for use in the compositions of the present invention include, but are not limited to, silica, calcined alumina, sodium bicarbonate, calcium carbonate, dicalcium phosphate and calcium pyrophosphate. If included, the abrasive is generally present at a concentration of from about 3 to about 50% by weight.

In some embodiments, surfactants are used in the compositions of the present invention. Suitable examples of surfactants include water-soluble salts of higher fatty acid monoglyceride monosulfates, such as the sodium salt of the monsulfated monoglyceride of hydrogenated coconut oil fatty acids, cocamidopropyl betaine, higher alkyl sulfates such as sodium lauryl sulfate, alkyl aryl sulfonates such as sodium dodecyl benzene sulfonate, higher alkyl sulfoacetates, sodium lauryl sulfoacetate, higher fatty acid esters of 1,2-dihydroxy propane sulfonate, and the substantially saturated higher aliphatic acyl amides of lower aliphatic amino carboxylic acid compounds, such as those having 12 to 16 carbons in the fatty acid, alkyl or acyl radicals, and the like. Examples of the last mentioned amides are N-lauroyl sarcosine, and the sodium, potassium, and ethanolamine salts of N-lauroyl, N-myristoyl, or N-palmitoyl sarcosine.

If included, surfactants are generally present at a concentration of from about 0.1 to about 5%, by weight of the composition. In some embodiments, the surfactant is present at a concentration of from about 0.5 to about 4%, by weight of the composition. In some embodiments, the surfactant is present at a concentration of from about 1 to about 3%, by weight of the composition. In some embodiments, the surfactant is present at a concentration of about 2%, by weight of the composition

For illustrative purposes, compositions of the present invention can be made by combining water, humectants, e.g. glycerin, sorbitol, polyethylene glycol in a conventional mixer until the mixture becomes a homogeneous gel phase. Into the gel phase are added the metal salt and, if included, abrasive(s) and the like. These ingredients are mixed until a homogeneous phase is obtained. Thereafter, a flavor component as described herein along with any thickeners, colorants, and surfactant are added and the ingredients are mixed at high speed in vacuum of about 20 to 100 mmHg.

Embodiments of the present invention are further described in the following examples. The examples are merely illustrative and do not in any way limit the scope of the invention as described and claimed.

### EXAMPLES

### Example 1

Flavor components according to the present invention (1-11) are shown below in Table 1, with amounts of components in weight %. These flavor components can be made via conventional methods.

TABLE 1

| | Flavor Component | | | | | | | | | | |
|---|---|---|---|---|---|---|---|---|---|---|---|
| | 1 | 2 | 3 | 4 | 5 | 6 | 7 | 8 | 9 | 10 | 11 |
| Ingredient | % w/w | | | | | | | | | | |
| L-menthol | 35 | 47 | 41 | — | 38 | 39 | 42 | — | — | 36 | 43 |
| N-ethyl-para- | — | 5.4 | — | 7.6 | — | 5.5 | 7.2 | 6.1 | — | 5.7 | 7.3 |
| menthan-3- | | | | | | | | | | | |
| carboxamide | | | | | | | | | | | |
| Anethole | 8.3 | 11.2 | — | — | 8.7 | 10 | 9.6 | — | — | 9.3 | 8.5 |
| Cinnamic aldehyde | 1.4 | 0.6 | 0.8 | 0.5 | 0.3 | 0.7 | 0.5 | 0.8 | 0.6 | 0.9 | 0.8 |
| Eugenol | 0.2 | 0.8 | 0.8 | 0.9 | 1.6 | 0.7 | 0.6 | 0.9 | 0.6 | 0.8 | 0.5 |
| Eucalyptol | 1.1 | 2.6 | 2.3 | 3.1 | 5.2 | 3.6 | 2.4 | 3 | 2.7 | 3.2 | 2.8 |
| Peppermint oil | 24.8 | — | — | 23.7 | — | 25 | 22.6 | — | — | 23 | 26.1 |
| Spearmint oil | — | 4.2 | — | — | 3 | 2 | — | 1.8 | — | 4.1 | 1.2 |
| Cornmint oil | 6.2 | — | — | 7.2 | — | 5 | — | 5.4 | 6.3 | 7.6 | — |

### Example 2

The formulations of an exemplary composition of the present invention (Composition I) and two comparative examples (Compositions X and Y) are described in Table 2 (below).

TABLE 2

| | Composition | | |
|---|---|---|---|
| | I | X | Y |
| Ingredient | % w/w | | |
| Water | 17.8 | 17.8 | 15.3 |
| Sodium saccharin | 0.3 | 0.3 | 0.3 |
| Sodium monofluorophosphate | 1.1 | 1.1 | — |
| Sodium fluoride | — | — | 0.24 |
| Triclosan | — | — | 0.3 |
| Zinc citrate trihydrate | 2 | 2 | — |
| Glycerin | 16.5 | 16.5 | 20 |
| PEG 600 | 3 | 3 | — |
| Propylene glycol | — | — | 0.5 |
| Sodium CMC | 0.6 | 0.6 | 1.1 |
| Carrageenan | — | — | 0.5 |
| Xanthan gum | 0.4 | 0.4 | — |
| Sorbitol (70% solution) | 17.8 | 17.8 | 20.9 |
| Tetrapotassium pyrophosphate | 2.4 | 2.4 | — |
| PVM/MA Co-polymer | 11.5 | 11.5 | 15 |
| Sodium hydroxide (50% solution) | 1.3 | 1.3 | 1.2 |
| Titanium dioxide | 1 | 1 | 0.75 |
| Abrasive silica | 20 | 20 | 18.5 |
| Thickening silica | 1.8 | 1.8 | 3 |
| Sodium lauryl sulfate | 1.5 | 1.5 | 1.5 |
| Flavor component | 1 | 1 | 1 |

Compositions I, X and Y were evaluated in a home use consumer acceptability study. The results of the study demonstrate that an exemplary flavor component of the present invention provided improved consumer acceptability over similarly formulated compositions using flavor components different from those described herein.

1. A flavor component for use in an oral care composition containing a metal salt, comprising:
a taste-masking agent comprising: cinnamic aldehyde; eugenol; and eucalyptol; and
one or more flavoring agents selected from: L-menthol; N-ethyl-para-menthan-3-carboxamide; anethole; peppermint oil; spearmint oil; corn mint oil; and a combination of two or more thereof.

2. The flavor component of claim 1, comprising greater than 0.1%, by weight, cinnamic aldehyde.

3. The flavor component of claim 1 or claim 2, comprising greater than 0.1%, by weight, eugenol.

4. The flavor component of any one of claims 1 to 3, comprising greater than 0.5%, by weight, eucalyptol.

5. The flavor component of any one of claims 1 to 4, wherein at least one of the one or more flavoring agents is L-menthol.

6. The flavor component of any one of claims 1 to 5, wherein at least one of the one or more flavoring agents is N-ethyl-para-menthan-3-carboxamide.

7. The flavor component of any one of claims 1 to 6, wherein at least one of the one or more flavoring agents is anethole.

8. The flavor component of any one of claims 1 to 7, wherein at least one of the one or more flavoring agents is peppermint oil.

9. The flavor component of any one of claims 1 to 8, wherein at least one of the one or more flavoring agents is spearmint oil.

10. The flavor component of any one of claims 1 to 9, wherein at least one of the one or more flavoring agents is corn mint oil.

11. The flavor component of any one of claims 5 to 10, comprising greater than 29.5%, by weight, L-menthol.

12. The flavor component of any one of claims 6 to 11, comprising greater than 4.5%, by weight, N-ethyl-para-menthan-3-carboxamide.

13. The flavor component of any one of claims 7 to 12, comprising less than 14.5%, by weight, anethole.

14. The flavor component of any one of claims 8 to 13, comprising less than 40.5%, by weight, peppermint oil.

15. The flavor component of any one of claims 9 to 14, comprising less than 5.5%, by weight, spearmint oil.

16. The flavor component of any one of claims 10 to 15, comprising greater than 4.5%, by weight; corn mint oil.

17. A flavor component for use in an oral care composition containing a metal salt, comprising:
greater than 29.5%, by weight, L-menthol;
greater than 4.5%, by weight, N-ethyl-para-menthan-3-carboxamide;
less than 14.5%, by weight, anethole;
greater than 0.1%, by weight, cinnamic aldehyde;
greater than 0.1%, by weight, eugenol;
greater than 0.5%, by weight, eucalyptol;
less than 40.5%, by weight, peppermint oil;
less than 5.5%, by weight, spearmint oil; and
greater than 4.5%, by weight, corn mint oil.

18. The flavor component of claim 17, comprising:
from about 30 to about 50%, by weight, L-menthol;
from about 5 to about 10%, by weight, N-ethyl-para-menthan-3-carboxamide;
from about 5 to about 14%, by weight, anethole;
from about 0.1 to about 1%, by weight, cinnamic aldehyde;
from about 0.1 to about 1%, by weight, eugenol;
from about 1 to about 5%, by weight, eucalyptol;
from about 20 to about 40%, by weight, peppermint oil;
from about 1 to about 5%, by weight, spearmint oil; and
from about 5 to about 10%, by weight, corn mint oil.

19. The flavor component of claim 17 or claim 18, comprising:
from about 35 to about 45%, by weight, L-menthol;
from about 5 to about 8%, by weight, N-ethyl-para-menthan-3-carboxamide;
from about 8 to about 12%, by weight, anethole;
from about 0.5 to about 0.9%, by weight, cinnamic aldehyde;
from about 0.5 to about 0.9%, by weight, eugenol;
from about 2 to about 4%, by weight, eucalyptol; and
from about 22 to about 27%, by weight, peppermint oil.

20. An oral care composition comprising:
the flavor component of any one of claims 1 to 19;
one or more metal salts selected from: a zinc salt; a calcium salt; a copper salt; an iron salt; a magnesium salt; a manganese salt; and a combination of two or more thereof; and
an orally acceptable carrier;
wherein the total concentration of said one or more metal salts is from about 0.01 to about 5%, by weight of the composition.

21. The composition of claim 20, wherein at least one of said one or more metal salts is a zinc salt selected from: zinc oxide; zinc sulfate; zinc chloride; zinc citrate; zinc lactate; zinc gluconate; zinc malate; zinc tartrate; zinc carbonate; zinc phosphate; and a combination of two or more thereof.

22. The composition of claim 20 or claim 21, wherein the zinc salt is selected from: zinc oxide; zinc citrate; zinc gluconate; zinc glycinate; zinc lactate; and a combination of two or more thereof.

23. The composition of any one of claims 20 to 22, wherein the zinc salt is selected from: zinc oxide; zinc citrate; and a combination of two or more thereof.

24. The composition of any one of claims 20 to 23, wherein the zinc salt is zinc citrate.

25. The composition of any one of claims 20 to 24, wherein the metal salt is present at a concentration of from about 0.1 to about 4%, by weight.

26. The composition of any one of claims 20 to 25, wherein the metal salt is present at a concentration of from about 1 to about 3%, by weight.

27. The composition of any one of claims 20 to 26, wherein the metal salt is present at a concentration of about 2%, by weight.

28. The composition of any one of claims 20 to 27, wherein the flavor oil component is present at a concentration of from about 0.5 to about 2.5%, by weight.

29. The composition of any one of claims 20 to 28, wherein the flavor oil component is present at a concentration of from about 0.8 to about 1.5%, by weight.

30. The composition of any one of claims 20 to 29, wherein the flavor oil component is present at a concentration of about 1%, by weight.

31. The composition of any one of claims 20 to 30, further comprising one or more components selected from: a fluoride ion source; a tartar control agent; a buffering agent; an abrasive; and a combination of two or more thereof.

32. The composition according to any one of claims 20 to 31, wherein at least one of said one or more components is a fluoride ion source selected from: stannous fluoride, sodium fluoride, potassium fluoride, sodium monofluorophosphate, sodium fluorosilicate, ammonium fluorosilicate, amine fluoride, ammonium fluoride, and a combination of two or more thereof.
